(19) United States
(12) Patent Application Publication
(10) Pub. No.: US 2009/0306528 A1
(43) Pub. Date: Dec. 10, 2009

(54) ADAPTIVE TEMPERATURE SENSOR FOR BREATH MONITORING DEVICE

(75) Inventors: James N. Curti, Bakersfield, CA (US); Kyle L. Adriance, Bakersfield, CA (US); Eric C. Land, Bakersfield, CA (US)

Correspondence Address: DAVIS & BUJOLD, P.L.L.C., 112 PLEASANT STREET, CONCORD, NH, 03301, US

(73) Assignee: SALTER LABS, Arvin, CA (US)

(21) Appl. No.: 12/134,787

(22) Filed: Jun. 6, 2008

(51) Int. Cl. A61B 5/087

(52) U.S. Cl. 600/537; 600/538

(57) ABSTRACT

A system and method for sleep monitoring, diagnosing and sensing temperature and pressure for a breathing cycle of a patient including a sensing device suitable for both nasal and oral breath monitoring for measuring respiratory air wave and airflow information during a sleep apnea diagnostic session and processing the acquired air wave and airflow breathing information for input to conventional polysomnography equipment.

### FIELD OF THE INVENTION

The present invention relates to a sleep monitoring and diagnosing system including a temperature sensing and pressure sensing device suitable for both nasal and oral breath monitoring for measuring respiratory air wave and airflow information during a sleep apnea diagnostic session and processing the acquired air wave and airflow breathing information for input to conventional polysomnography equipment. The temperature and pressure sensing devices can be used individually or concurrently and where utilized together have a structural and signal based relationship which facilitates obtaining a verified output representative of the patients breathing patterns.

### BACKGROUND OF THE INVENTION

Sleep apnea (SA) is a common disorder observed in the practice of sleep medicine and is responsible for more mortality and morbidity than any other sleep disorder. SA is characterized by recurrent failures to breathe adequately during sleep (termed apneas or hypopneas) as a result of obstructions in the upper airway.

Apnea is typically defined as a complete cessation of airflow. Hypopnea is typically defined as a reduction in airflow disproportionate to the amount of respiratory effort expended an/or insufficient to meet the individual's metabolic needs. During an apnea or hypopnea, commonly referred to as a respiratory event, oxygen levels in the brain decrease while the carbon dioxide (CO2) levels rise, causing the sleepier to awaken. The heart beats rapidly and blood pressure rises to levels (up to 300 mm Hg). The brief arousals to breathe are followed by a return to sleep, but the apneas may recur over 60 times per hour in severe cases.

SA is a serious, yet treatable health problem for individuals worldwide. Published reports indicate that untreated SA patients are three to five times more likely to be involved in industrial and motor vehicle accidents that have impaired vigilance and memory. Studies show that more than 15% of men and 5% of women over the age of 30 and up to 30% of mend and women over the age of 65 suffer from SA. SA during pregnancy is associated with hypertension and a risk of growth retardation in the fetus. Current estimates reveal that over 90% of individuals with moderate to severe SA remain undiagnosed.

The current standard for the diagnosis of SA is called polysomnography (PSG), that is administered and analyzed by a trained technician and reviewed by a Board Certified Sleep Specialist. The limited availability of sleep centers coupled with the high capital expense to add capacity has resulted in a growing number of patients awaiting their PSG.

A conventional full overnight PSG includes recording of the following signals: electroencephalogram (EEG), sub-mental electromyogram (EMG), electroculogram (EOG), respiratory airflow (oronasal flow monitors), respiratory effort (plethysmography), oxygen saturation (oximetry), electrocardiography (ECG), snoring sounds, and body position. These signals are considered the “gold standard” for the diagnosis of sleep disorders in that they offer a relatively complete collection of parameters from which respiratory events may be identified and SA may be reliably diagnosed. The RR interval, commonly referred to as beats per minute, is derived from the ECG. Body position is normally classified as: right side, left side, supine, prone, or up (or sitting erect). Typically, the microphone is taped over the pharynx and the body position sensor is attached over the sternum of the patient's chest. Each signal provides some information to assist in the visual observation and recognition of respiratory events.

Collapse of the upper airway is identified when the amplitude of the respiratory airflow and the effort signals decrease by at least 50%, snoring sounds either crescendo or cease, and oxygen desaturation occurs. A respiratory event is confirmed (i.e., desaturation not a result of artifact) by the recognition of an arousal (i.e., the person awakens to breathe), typically identified by an increase in the frequency of the EEG, an increase in the heart rate, or changing in snoring patter. The remaining signals assist in determining specific types of respiratory events. For example, the EEG and EOG signals are used to determine if a respiratory event occurred in non-rapid eye movement (NREM) or rapid eye movement (REM) sleep. The position sensor is used to determine if an airway collapse occurs only or mostly in just one position (typically supine).

A reduction or absence of airflow at the airway opening defines sleep-disordered breathing. Absent airflow for 10 seconds in an adult is apnea, and airflow reduced below a certain amount is a hypopnea. Ideally one would measure actual flow with a pneumotachygraph of some sort, but in clinical practice this is impractical, and devices that are comfortable and easy to use are substituted. The most widely used are thermistors placed in front of the nose and mouth that detect heating (due to expired gas) and cooling (due to inspired air) of a thermally sensitive resistor. They provide recordings of changes in airflow, but as typically employed are not quantitative instruments. Currently available thermistors are sensitive, but frequently lag or have a delay in response time relative to pressure sensors and pressure transducers. Also, if they touch the skin, they cease being flow sensors. Measurement of end tidal CO2 is used in some laboratories to detect expiration to produce both qualitative and quantitative measures of a patients breath.

An alternative method is to measure changes in pressure in the nasal airway that occur with breathing. This approach provides an excellent reflection of true nasal flow. A simple nasal cannula attached to a pressure transducer can be used to generate a signal that resembles that obtained with a pheumatachygraph. It allows detection of the characteristic plateau of pressure due to inspiratory flow limitation that occurs in subtle obstructive hypopneas.

An obstructive apnea or hypopnea is defined as an absence or reduction in airflow, in spite of continued effort to breathe, due to obstruction in the upper airway. Typical polysomnography includes some recording of respiratory effort. The most accurate measure of the effort is; a change in pleural pressure as reflected by an esophageal pressure monitor. Progressively more negative pleural pressure swings leading to an arousal have been used to define a “Respiratory Effort Related Arousal” (RERA), the event associated with the so-called “upper Airway Resistance Syndrome”. However the technology of measuring esophageal pressure is uncomfortable and expensive, and rarely used clinically. Most estimates of respiratory effort during polysomnography depend on measures of rib cage and/or abdominal motion. The methods include inductance or impedance plethysmography, or simple strain gages. Properly used and calibrated, any of these devices can provide quantitative estimates of lung volume and abdominal-rib cage paradox. However calibrating during an overnight recording is very difficult and as a practical matter is almost never done. The signals provided by respiratory system motion monitors are typically just qualitative estimates of respiratory effort.

Pressure sensing devices are currently available and used during a sleep diagnostic session to detect changes in respiratory air pressure and/or airflow to confirm whether or not a patient is breathing and to gather other breathing information from the patient. Accurate modeling of the patient's breathing cycle is limited by the use of only pressure sensors as the placement of sensors and system failures can cause false readings or pressure offsets that must be adjusted to properly model the breathing cycle.

Combining pressure sensor measurements with temperature sensor measurements can improve breath monitoring and modeling leading to a more accurate diagnosis and more quickly determine a patient breathing failure by utilizing temperature monitors directly positioned at the nasal and oral breathing passages of the patient. Additionally, in using a temperature sensor for breath monitoring it is generally necessary to test the electrical leads and circuit components of the temperature sensing device to insure that all of the electrical leads and components are, in fact, operational and not faulty.

In addition, conventional test circuitry typically is completely separate from the temperature sensing device and this leads to further difficulties such as the test circuitry being either misplaced, lost, may have insufficient electrical power, etc., thereby rendering it difficult to test the pressure sensing device prior or during use.

### SUMMARY OF THE INVENTION

It is an object of the invention to provide a system including an apparatus and method for monitoring patient breathing through a temperature sensor and pressure sensor adapted for use with a nasal and oral cannula.

It is a further object of the invention to provide a method of securing a temperature sensor to a nasal and oral cannula such that the temperature sensor can be positioned directly at the outlet of the nares of the patients nose and adjusted to properly position the sensors in the air flow from the patient's mouth and nose and out of contact with the patients skin.

Another object of the invention is to provide an electronic circuit for the temperature sensors that includes a test circuit for determining the continuity of the temperature sensor circuit as a whole. The electronic circuit also has connections to an external microprocessor or controller to measure and accurately model patient breathing patterns based on the temperature and pressure data so as to provide a diagnosis for sleep apnea or alternatively to provided a basis for a determining proper gas and oxygen delivery to a patient.

Another object of the present invention is to facilitate ease of use of a coupled nasal cannula and temperature sensing device whereby the temperature sensing device mounts securely to portion of the cannula and the structure of the mount and temperature sensing device permits relative adjustment of the sensors into position to properly align with the patients nasal and oral expiration and inspiration, i.e. air flow.

Another object of the present invention is to provide test circuitry which is integrated directly into the signal temperature sensing device and readily allows the temperature sensing device to be quickly and conveniently tested prior to and during use of the temperature sensing device including a visual or audible indicator which indicates the continuity of the circuit and which test circuit does not continuously use power except when actuated by a user to test the circuit.

Yet another object of the present invention is to provide test circuitry in which the integrity of all of the internal circuitry of the temperature sensing device can be quickly and conveniently checked, by utilizing an internal battery powered circuit, to insure that there is adequate electrical conductivity for all of the internal circuitry and that none of the internal circuits are open, e.g., no electrical short is contained in any of the internal circuits.

The present invention relates to an airflow and temperature sensing device adaptive to a cannula for receiving respiratory breathing information from a patient to be monitored, the temperature sensing device comprising: a nasal breath monitor and an oral breath monitor configured as a series of thermistors inserted within an insulating sleeve and arranged in a T-shape form so as to adapt to connection with the rounded tubular surface of a nasal and oral cannula. Each thermistor is a temperature sensing device and is connected to wire leads that exit the insulating sleeve at each extension of a nares support frame within the nasal breath monitor. The T-shaped sensor configuration includes a right frame branch and a left frame branch that each extend from opposing sides of a central point to form an adjustable nares bridge. The nares bridge is flexible and allows movement of each of the branches in essentially a 360 degree freedom of movement range to provide for proper alignment of the thermistors mounted within each branch with the nasal air flow of the patient for proper monitoring.

An oral support branch extends from the central point to form the oral breath monitor. An oral temperature sensor is mounted within the oral support branch spaced from the adjustable nares bridge. Manipulating the adjustable oral branch the oral sensor can be moved axially of laterally, i.e. 360 degrees to properly align the oral temperature sensor with the oral breath of the patient for proper monitoring.

In one embodiment of the invention, each temperature sensor is a thermistor with negative temperature coefficient characteristics that exhibit a decrease in electrical resistance as temperature increases and increase in electrical resistance as temperature decreases. Changes in temperature within a range of 1° C. to 2° C. and more specifically within a 1° C. will change the resistance of the thermistor sensor and cause an increase or decrease in current within an external temperature sensor or respiratory airflow detection circuit. In attaching the temperature sensor to a nasal and oral cannula with the use of a special mounting holster integrated within the cannula the breathing cycle of a patient can be monitored. On exhalation by the patient there will be an increase temperature of the air immediately at the base of the nasal outlet or nares and at the oral outlet of the mouth. This increase in temperature will decrease the resistance of the temperature sensor thermistors causing an electrical change within the respiratory airflow detection circuit. In one embodiment, this electrical change creates a change in frequency within a capacitive filter circuit generating a signal emission that is read by a microprocessor that tracks the amplitude and frequency of each thermistor resistance change. Each exhalation and inhalation of the patient is directly tracked by the close proximity of the temperature sensor to the nares and oral cavity of the patient.

Temperature modeling of the breathing cycle could supplement the commonly used pressure sensor breath cycle modeling to better indicate aberrations within the cycle and more reliably track changes that are related specifically to the breathing physiology of the patient and not external limitations of the monitoring system. Temperature sensors directly at the patient's nose and mouth more accurately detect changes and more quickly detect the stoppage of breathing by the patient providing for the use of the external resistance change to activate an alarm signal to indicate the patient is in distress.

The use of sensors for monitoring breathing of patients requires that the circuitry within the system be operational and free from faults prior and during use. The present invention includes test circuitry that identifies faults in the thermistors, the thermistor leads and the internal circuit components of the respiratory airflow detection circuit. No external test equipment is required to safely and easily test if the leads are free from shorts or opens and to determine that the thermistors and other circuitry components are operational. In one embodiment the external leads from the thermistors and nares support frame are connected to test circuitry that can be activated to test continuity and powered operation within the system by pressing a test button and visually acknowledging a lighted LED indicator that confirms circuit operation is properly functioning. A failure of the LED to light indicates a system fault that must be investigated prior to use.

The present invention relates to a temperature sensing device for coupling to a cannula and receiving respiratory breathing information from a patient to be monitored. The temperature sensing device has an internal test circuit for testing an integrity of all electrical leads and circuit components prior to use for ensuring that the temperature sensing device is operational.

The present invention also relates to a method of using a cannula to receive respiratory breathing information from a patient to be monitored, the method comprising the steps of: using a temperature sensing device comprising an support frame with adjustable bride supports and temperature sensors mounting along to support frame for receiving the respiratory breathing information from the patient to be monitored; processing the received respiratory breathing information from the patient and outputting, a signal indicative of the sensed breathing cycle of the patient; accommodating a respiratory airflow detection circuit within an exterior housing for processing the received respiratory breathing information from the patient and outputting, a signal indicative of sensed airflow of the patient; and testing an integrity of the electrical leads, temperature sensors and circuit components via an internal test circuit, prior to use of the temperature sensing device, to ensure that the temperature sensors for breath monitoring are operational.

### BRIEF DESCRIPTION OF THE DRAWINGS

The invention will now be described, by way of example, with reference to the accompanying drawings in which:

FIG. 1 is a flow diagram representation of a the present invention within a breath monitoring system;

FIG. 2 is a graph illustrating a flow rate profile of the breathing cycle of a patient combining pressure sensor and temperature sensor data;

FIG. 3A is a diagrammatic representation of the temperature sensor of the present invention;

FIG. 3B is a perspective view of an embodiment of the pressure and temperature sensor mounted together without an oral pressure sensing prong;

FIG. 3C is a perspective view of an embodiment of the pressure and temperature sensor mounted together with an oral pressure sensing prong;

FIG. 4A is representation of the cannula and temperature sensor and associated initial arm angle of the appertaining arms of the cannula;

FIGS. 4B and 4C are representations of the cannula and temperature sensor and associated adjacent angles of the appertaining arms of the cannula;

FIG. 5 is a circuit schematic diagram of the respiratory airflow detection circuit with test circuitry to test operational functionality of the temperature sensor;

FIG. 6 is a front perspective view of a cannula of a first embodiment of the present invention used to support the temperature sensor;

FIG. 7 is a rear view of the cannula of the first embodiment used to support the temperature sensor;

FIG. 8 is a side view of the cannula of the first embodiment used to support the temperature sensor via the holster of the cannula;

FIG. 9 is a perspective view of a second described embodiment of the cannula having an oral pressure sensing prong extending therefrom;

FIG. 10 is a side view of the cannula of the second embodiment of the invention used to support the temperature sensor shown therewith;

FIG. 11 is a bottom perspective view of the cannula of the second embodiment used to support the temperature sensor; and

FIG. 12 is a rear view of the cannula of the second embodiment used to support the temperature sensor.

### DETAILED DESCRIPTION OF THE INVENTION

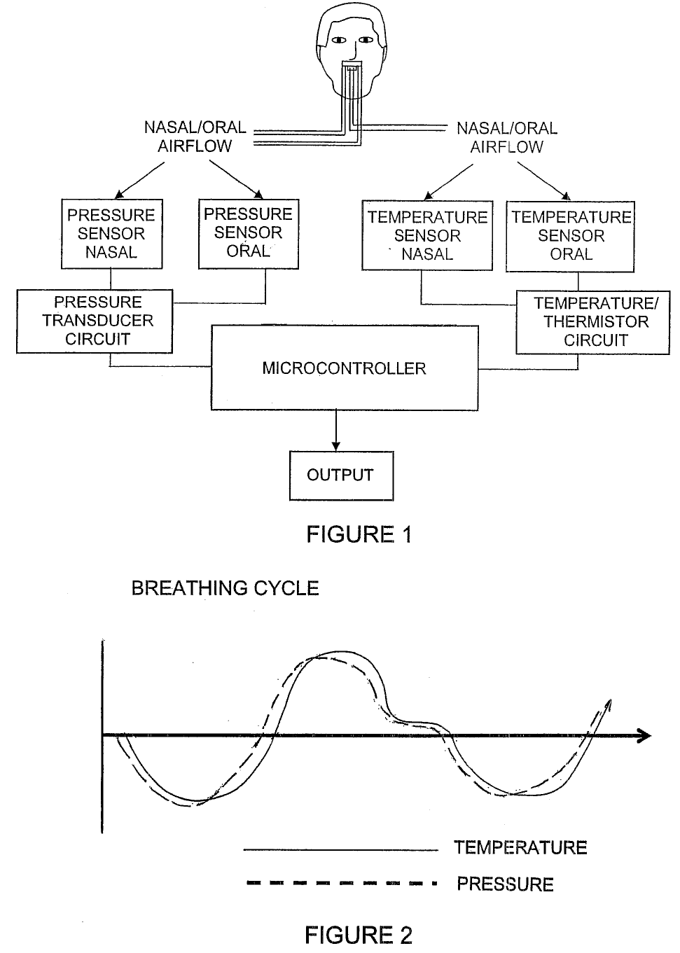

The present invention is directed to an apparatus and method for monitoring and modeling a patient's breathing according to both pressure and temperature measurements. As seen in FIG. 1, from oral and nasal airflow of a patient oral and nasal temperature measurements are obtained according to temperature changes measured by a thermistor during the exhalation and inhalation interval of a patient during a sleep diagnostic session. A temperature sensor, generally a thermistor although other types of thermocouples and temperature sensors could be used as well, is positioned adjacent the nares (nostrils) of the patients nose (nasal temperature sensing) and adjacent the patients mouth (oral temperature sensing). An output signal from the temperature sensor(s) is conditioned by a thermistor circuit and sent to a micro controller to be processed into acquired air wave and airflow breathing data for input to conventional polysomnography equipment which produces an output representation of the patients breathing cycle generally as a qualitative, viewable waveform.

A pressure sensor is also used in the system in conjunction with the temperature sensor. The pressure sensor like the thermistor is a non-invasive alternative for measuring nasal and oral airflow of a patient during the diagnostic studies. A pressure sensor is generally the preferred method of determining nasal air flow since the nasal prongs of the cannula are situated essentially inside the nares of the patients nose and directly in the flow path of nasal inspiration and expiration. It follows that nasal pressure sensing often achieved with a pressure transducer is generally a more accurate method of assessing hypopneas in real time, critical to the accurate diagnosing of a patient.

If a patient breaths through their mouth on the other hand it is more difficult to obtain an accurate pressure measurement based on inspiration and expiration through the mouth. Because of the size of a patients mouth in general, it is difficult to align an oral prong or cannula opening an an appropriate position to obtain the oral inspiration and expiration. For example a person may breath out the side of their mouth and thus an oral prong located in the center of the mouth for pressure sensing may not receive adequate breathing flow to properly determine pressure. In the case of a mouth breather like this, the temperature sensor with an oral thermistor may provide the best response using the temperature differential between the ambient air and whatever portion of the patients breathing is obtained.

To determine an accurate wave form of the patient's breathing a nasal cannula is generally used by the patient which is then connected to a pressure sensor for example a sensitive pressure transducer. The pressure transducer emits a signal which is proportional to the flow and this signal is processed by the micro controller to generate a respiratory waveform signal which indicates the fluctuations in pressure caused by inspiration and expiration of the patient. In the present system a temperature sensor may also be used with the cannula, or mask in the case of titration, to provide further accuracy in determining breathing cycle data and an accurate wave form.

In general, and as discussed in further detail below, in order to most effectively determine an actual accurate wave form including the most accurate amplitude as well as frequency, i.e. breaths per minute, the present embodiment of the system includes a thermistor(s) as the temperature sensor for obtaining the oral and nasal temperature changes of a patients inspiration and expiration is adapted to be affixed to a nasal and oral cannula. The cannula is used as described above to obtain the nasal and oral airflow and derived pressure changes in the patients breathing which, along with the data obtained by the thermistor can then be compared to obtain the most accurate waveform and most precise monitoring and diagnosis of a patient's respiratory airflow and breathing cycles including confirmation of distress signals from hypopneas or apnea events.

FIG. 1 is a basic flow chart of an embodiment of a temperature and pressure sensor breath monitoring system for providing conformational data of changes or aberrations within a patient's breathing cycle from a nasal pressure sensor and oral pressure sensor as well as a nasal temperature sensor and oral temperature sensor. The attachment of the temperature sensor and thermistors to the cannula provides that the thermistors are adjacent the oral and nasal passages of the patient to obtain an accurate temperature change in concurrence with the nasal and oral inlets to the cannula which receive the air flow indicative of pressure changes which effect the pressure sensor. The nasal pressure sensor is provided in conjunction with the oral pressure sensor via the cannula to provide a pressure signal to the micro controller, and the nasal temperature sensor along with an oral temperature sensor via a thermistor is connected to the micro-controller to supply a further temperature change signal to the micro-controller. This system therefore provides a pressure and temperature signal from each breathing cycle to the microprocessor or controller and can there be accumulated, processed and provided as a breathing pattern output for diagnosis and treatment purposes.

FIG. 2 shows an example of a breathing pattern output derived from the acquired temperature and pressure data of the patient's breathing cycle. Pressure data is collected from the cannula and pressure sensor on the one hand, and on the other temperature data is also collected from the oral and nasal temperature sensors over a period of time to track the patient's breathing cycle. When both the pressure and temperature sensors are plotted together as seen in FIG. 2 it becomes apparent despite any lag time in the temperature measurement and response where potential anomalies or errors may exist in the respective temperature and pressure sensors and signals, and also that the system can more reliably detect apnea, hyopopnea and other subtle flow limitations where both pressure and temperature signal outputs can be concurrently determined from a patients baseline oral and nasal breathing pattern.

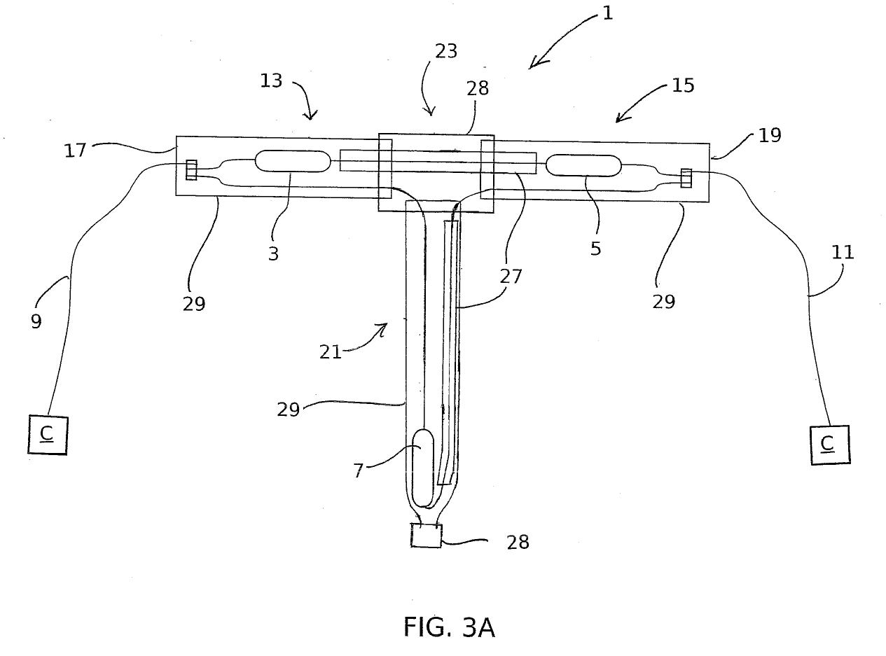

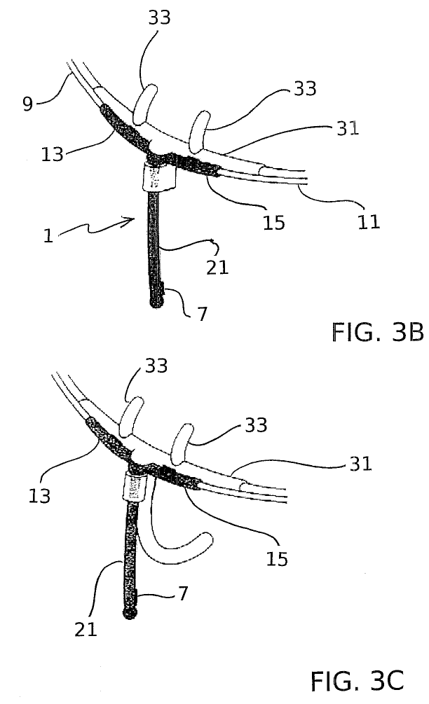

Turning to FIGS. 3A, 3B and 3C the temperature sensor 1 of the embodiment shown here is a triad, i.e. three, thermistors 3, 5 and 7 comprising a first nasal thermistor 3 in series with a second nasal thermistor 5 on a nasal circuit, and an oral thermistor 7 that is positioned along an oral circuit connected in parallel and structurally aligned perpendicular to the nasal circuit and first and second nasal thermistors 3 and 5. A first and second leads 9 and 11 are connected to the respective circuit junctions of the nasal and oral circuits to send the resistivity change to a conditioning circuit C described in further detail below.

The temperature sensor 1 including the thermistors is formed in a T-shape configuration with the first nasal thermistor 3 located in a left branch 13 of the sensor 1. The second thermistor 5 positioned in the right branch 15 of the sensor 1, and the oral thermistor 7 located in the lower branch of the T-shaped sensor. When properly positioned on the cannula and on the face of a patient the left and right branches 13, 15 extend in each lateral direction under the nasal septum of the patients nose to respective free ends 17, 19 so that each of the nasal thermistors 3, 5 are positioned directly adjacent the opening to each respective left and right nares of the patients nose.

The left and right branches 13, 15 form a rigid but flexible bridge that provides structurally stable but flexible support to allow for each left and right branches 113, 15 to be adjusted, i.e. bent, manipulated, curved or articulated into an alternative position. Although the branches are shown here as being linearly aligned, the flexibility of the branches 13, 15 permits non-linear alignment as seen in subsequent figures. This non-linear flexibility facilitates aligning and maintaining the respective right and left nasal thermistors 3, 5 with the patients right and left nares and does so in conjunction with the nasal prongs of the cannula supporting the temperature sensing device. It is also to be appreciated that there do not necessarily have to be two thermistors 3, 5 in the bridge, but that there could also be a single thermistor located in the bridge which could be aligned with one of the patients nares, or even between the patients nares.

Similarly, a lower branch of the T-shaped sensor extends perpendicularly downwardly relative to the flexible bridge and is also adjustable, flexible and manipulatable such that the lower branch 21 which includes the oral temperature circuit and oral thermistor 7 provides the same rigidity and maleability to structurally support the oral thermistor adjacent the patients mouth. In the case of each branch 13, 15 and 21, the branches can independently arranged with respect to one another about the center joint 23. In other words each branch is radially flexible in a 360 rotational manner about the center joint 23, and each branch is also axially flexible, i.e. bendable along its longitudinal axis to ensure that the oral thermistor 7 is not only placed in an appropriate position adjacent the patient's mouth so that it is fully in the path of inspiration and expiration, but also can be adjusted so as not to touch any part of the patients mouth, skin or face.

The T-shape configuration of the temperature sensor 1 is important because by its very nature the T-shape defines three (3) independent branches 13, 15 and 21 extending from a center joint 23 to three (3) free ends. The left and right upper branches each define a left and right free end 17, 19 and the depending prong 29 also defines its own respective lower free end. With each branch extending in this manner from the center joint 23 to the respective free ends 17, 19 and 25 respectively, consequently each branch 3, 5, and 7 and associated thermistor can be independently adjusted, bent or configured to a desired shape or configuration independent of one another. By way of example, the left and right branches 13, 15 may be bent in a manner to curve laterally in cooperation with the curved shape of the cannula or the curved skin and face surface of the patient as seen in FIGS. 3B and 3C. This allows each thermistor in the sensor to be aligned in the most direct flow path of the nasal airflow passing through the patients nares. Similarly, but independently of the left and right branches 13, 15 the lower branch 21 may be curved, bent or manipulated so as to most effectively position the oral 7 thermistor in the most advantageous position to receive the oral temperature change from the patient's oral airflow. Also, by appropriately arranging the lower branch 21 independent of the left and right branches 13, 15 it can be assured that the lower branch 21 and the oral thermister does not contact the patients skin and thereby adversely influence the response of the thermistor to the patients oral airflow.

This independent flexibility of the lower branch 21 is critical because if the oral thermistor 4 touches the skin or face of the patient, the thermistor will be effected by the body and skin temperature in addition to any temperature changes caused by the patient's breathing. Also, the ability to bend and manipulate the lower branch 21 in what is essentially a 360 degree manner ensures that the oral thermistor 7 is placed in the most direct path of the patients inspiration and expiration flow. While the flow path of inspiration and expiration generally does not vary significantly through the nares of the nose because of the relative smaller size of the nare openings as compared to the mouth, and the flow rate of a patients breathing, the mouth is much larger than the nares and a patient may breath out the side, top or bottom of their mouth. Thus, the ability to radially and axially articulate and maintain the lower branch 21 and hence the oral thermistor 7 in a region where the patients most direct oral inspiration and expiration is occurring is critical to obtaining an appropriate and accurate reading and response of oral expiration and inspiration. This rigid flexibility of the temperature sensor and adjustments thereof relative to the nares and mouth permits proper positioning and configuring of the temperature sensor to align and match the proper physical characteristics of patients independently of the nasal and oral prongs of the cannula to which the sensor 1 is attached.

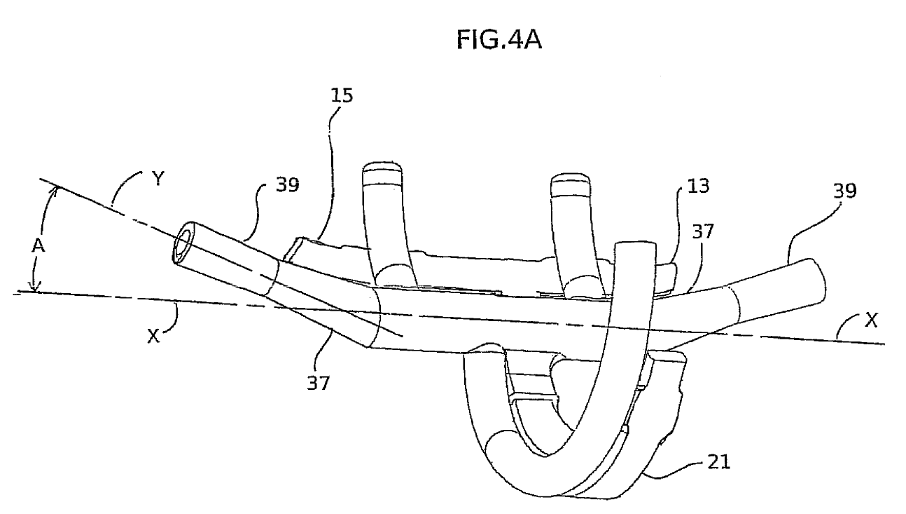

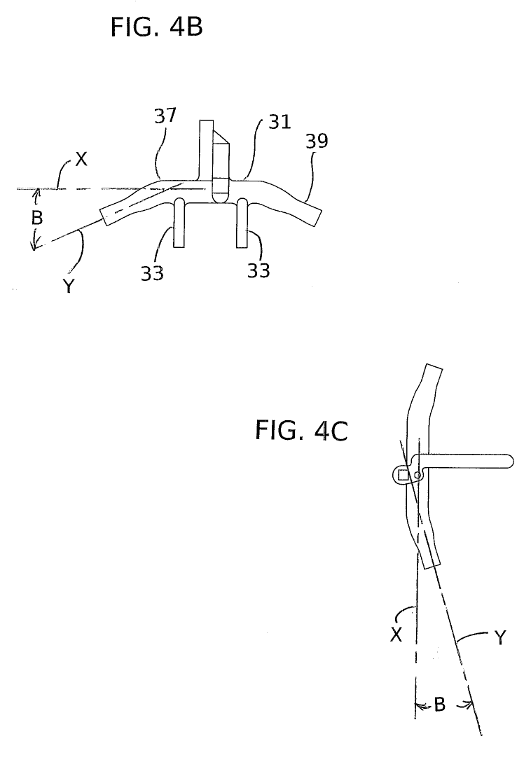

The ability to independently position the branches 13, 15 and 21 relative to the fixed orientation in which the center joint 23 of the temperature sensor 1 is held with respect to the cannula is also important in regards to the shape of the cannula 31 and cannula body 35. In an embodiment of the present invention, the cannula body 35 extends for a portion of its length along a main x-axis as best seen in FIGS. 4A, 4B and 4C. Elbows 37 are formed at either end bending in a 3-dimensional sense to define arms 39 extending along a y-axis. As explained more fully in U.S. Pat. No. 4,106,505, incorporated herein by reference, the y-axis extending along the length of each arm 39 intersect a horizontal plane defined by the x-axis of the main body 35 at an acute angle A from above the horizontal plane as seen in FIG. 4A. In FIGS. 4B and 4C the forward (towards the patients face) extension of the arms 39 defines an acute angle B of intersection between the y-axis and horizontal x-axis. The independent flexibility of each branch 13, 15 and 21 of the temperature sensor 1 ensures that the branches may be suitably positioned, and retained in such a position, where the branches not only conform to this described shape of the cannula body 35 but also where the nasal and oral thermistors can be best positioned relative to the cannula to receive the necessary air flow and also avoid touching the patients face.

It is to be appreciated that not all the branches 13, 15 and 21 are necessarily the same length. For example as discussed in further detail below, the temperature sensor 1 may be offset from a centerline of the cannula so that the left and right branches 13, 15 might be different lengths relative to the center joint 23 of the sensor 1 to properly position the respective thermistors 3, 5 adjacent the nasal prongs 33 and in the patients nasal airflow. Alternatively, where the branches 13, 15 are the same length the thermistors may be spaced different distances from the center joint 23 of the sensor 1 so that they align adjacent the nasal prongs 33 and in the nasal air flow of the patient. Clearly, the lower branch 21 may be longer than the upper branches 13, 15 to extend from the center joint 23 to an appropriate position in the oral air flow of the patient.

The nasal and oral thermistors 3, 5 and 7 and their respective circuits and wire leads 9, 11 shown in FIG. 3A may be joined in any manner known in the art for example soldering, taping, brazing or welding and protected and insulated by applying an inner layer of heat-shrink tubing 27 to protect and insulate these joints and connections. An outer layer of heat shrink material 29 may be applied over the circuits, joints, leads and thermistors as well to provide some level of insulation from the environment without degrading the response of thermistors and circuits. Also, any portions of the temperature sensor circuit not covered by the heat shrink material may be sealed with a non-conductive sealant or fixative for example a silicone polymer as layer 28, or some such similar non-conductive material to entirely seal the temperature sensor circuit from contact with ambient air. The center joint 23 of the T-shaped temperature sensor 1 may for example be sealed with the layer 28 to provide not only sealing and insulation of the circuit but also define a relatively rigid reference point from which each of the extending left and right branches 13, 15 and lower branch 21 extend and can be independently adjusted relative thereto.

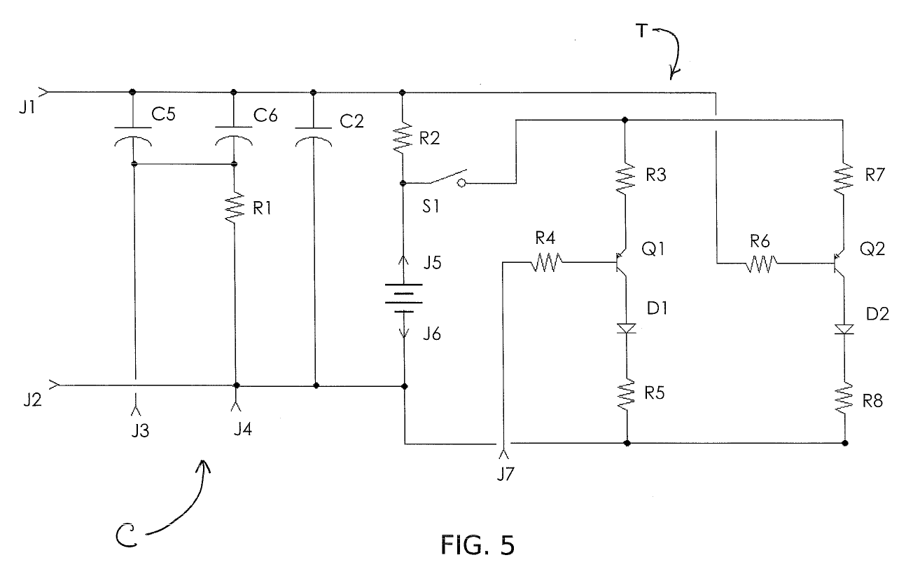

The airflow temperature sensor 1 can be a negative temperature coefficient (NTC) thermistor exhibiting decreasing electrical resistance with increases in environmental temperature and increasing electrical resistance with decreasing temperature. By way of example, the nasal temperature circuit as shown in FIG. 3A may have thermistors 3 and 5 with a resistance of 5 k each, while the oral thermistor 7 in parallel may be a 10 k resistance. In another embodiment all the thermistors could be arranged in series as 10 k resistance, particularly where a more substantial power supply is provided besides a small DC battery as discussed in regards to FIG. 5 below. A larger power supply would permit higher resistance to be used through the circuit and thus greater range of responsiveness to any temperature differential.

The temperature sensor 1 is provided with a left external lead 9 and a right external lead 11 which connect to a respiratory temperature detection circuit C having a test circuit as shown in FIG. 4. The respiratory airflow detection circuit C determines the change in temperature across the thermistor(s) based on the proportional change of a voltage divider in the circuit. The test circuit T ensures that the continuity of the circuit is maintained and can be monitored and readily ascertained at any desired time by pressing a button and without maintaining a diode or indicating light on at all times.

FIG. 5 is a schematic of the respiratory temperature detection circuit wherein the left external lead 9 is an input at J1 and right external lead 11 is input at J2. Power is applied to the circuit C; via a battery, for example a 3 volt coin cell connected to J5 (Pos) and J6 (Neg). Thermally equilibriating a change in temperature across the thermistors in the temperature sensor 1 will cause the voltage divider voltage to change proportionally with temperature at the junction of R2 and the thermistor lead terminal J1. If the rate of change in temperature is within a passband then the voltage can be measured at the head box leads.

The resistors and capacitors form a band pass filter with the combination of R2 and C2 forming a low pass filter with a cutoff frequency of around 42 Hz and the combination of C5 plus C6 and R1 forming the high pass filter with a cutoff frequency of around 0.066 Hz.

The capacitors C5 and C6 with resistor R1 and the resistive inputs of the temperature sensors through J1 and J2 form a filter capacitive circuit that generates frequency changes as the resistance changes within the thermistors of the temperature sensors on each inhalation and exhalation of the patients breathing cycle. An output analog signal is generated and fed to J3 and J4 to a microprocessor or other controller to model the patient's breathing cycle or to compare the signal to other breath monitors such as a pressure sensor output of oral or nasal breath as shown in FIGS. 1 and 2.

FIG. 5 also includes the test circuit T that tests the integrity of lead lines 9 and 11 connecting to J1 and J2, and the circuit components of the respiratory airflow detection circuit. The test circuit T includes a switch S1 that when closed creates a closed circuit of all components. Power is applied to the transistors circuits when the momentary switch S1 is closed. The D1 LED will light if a white or black head box lead is plugged into the J7 lead tester jack and S1 is closed verifying the integrity of the head box lead. The D2 LED will light when S1 is closed verifying the integrity of the thermistor leads. A failure within leads, connections or circuit components would fail to light a each test indicator at D1 and D2 and would identify an problem within the circuit.

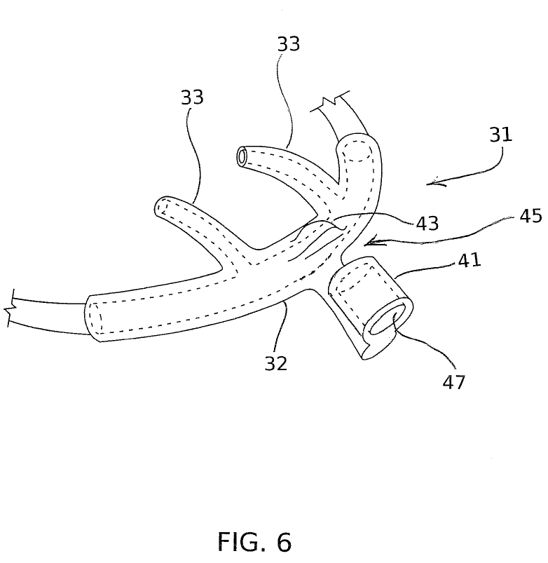

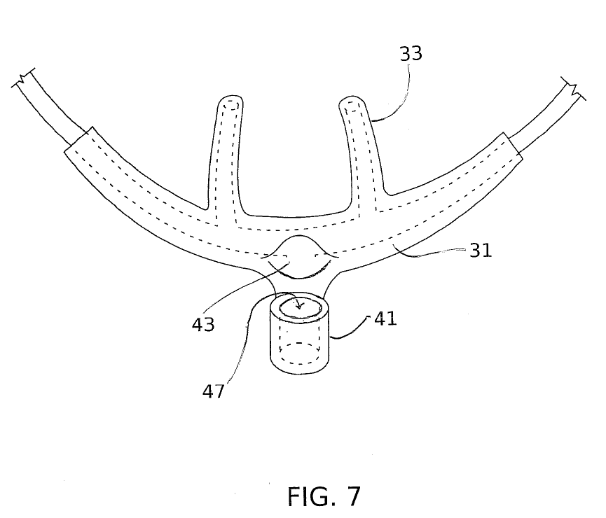

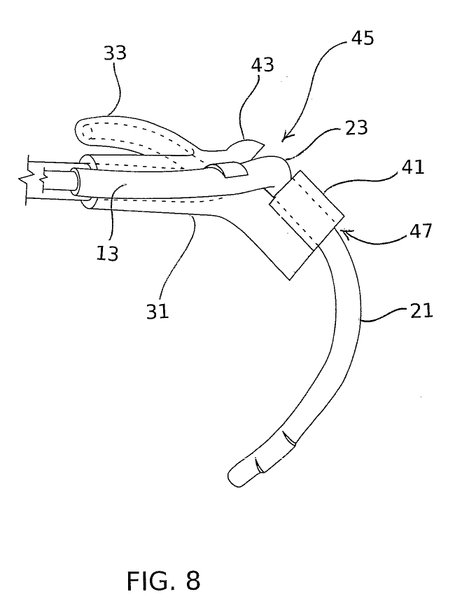

FIGS. 6, 7 and 8 detail a cannula 31 for use in the presently described system in conjunction with the above described temperature sensor 1 and circuit C. The cannula 31 includes a main cannula body 32 which is hollow having a first and second ends defining respective openings through which air and gas are delivered or received generally through a pair of nasal prongs 33 as are known in the art for receiving exhalation gases and/or supplying oxygen to the patient. The cannula 31 of this embodiment is further provided with an integral receiving holster 41 and stop portion 43 which defines a receiving notch 45 therebetween. The holster 41 is integrally connected with the body 32 of the cannula 31 and provided with a sensor passage 47. The sensor passage 47 may be of any particular shape, and does not even have to be entirely enclosed, i.e. as a cylinder, but is sized so as to receive a portion of the temperature sensor 1, namely the lower branch 21 which enters into the passage 47 and is frictionally retained therein.

During use as seen in FIG. 6, the lower branch 21 is pushed into the sensor passage 47 so that the oral thermistor 7 passes into and through the passage 47 and so extends out the bottom end of the passage 47. The lower branch 21 is pushed through the passage 47 until the extension of the left and right branches 13, 15 of the pressure sensor 1 abut a top end of the passage 47 and accordingly situate the center joint 23 of the T-shaped sensor snugly in the notch 45 between the stop portion 45 and the holster 41. The stop portion 43 which is also integrally connected with the body of the cannula 31, extends outward therefrom to an approximately the same dimensions of the holster 41. The space or notch 45 defined between the upper stop 43 and the passage 47 thus closely receives and holds the center joint 23 but is sufficiently flexible to facilitate the insertion and removal of the pressure sensor 1 into the passage 47 of the holster 41.

Once the T-shaped temperature sensor 1, as seen in FIG. 6 is inserted into the sensor passage 47, the branches 13, 15 and 21 may be independently manipulated in order to provide the appropriate alignment and curvature to these branches and their free ends as independently and as necessary in order to facilitate the most reliable data collection as previously described.

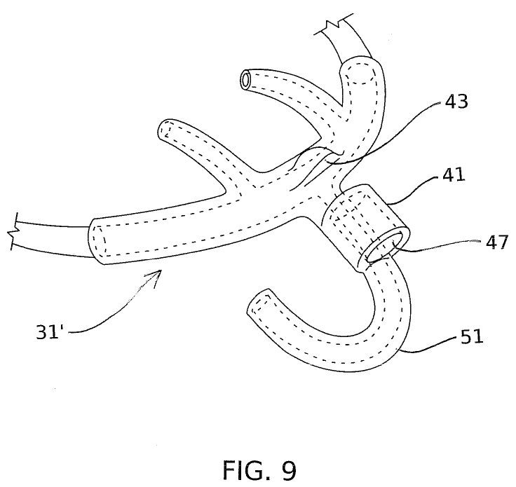

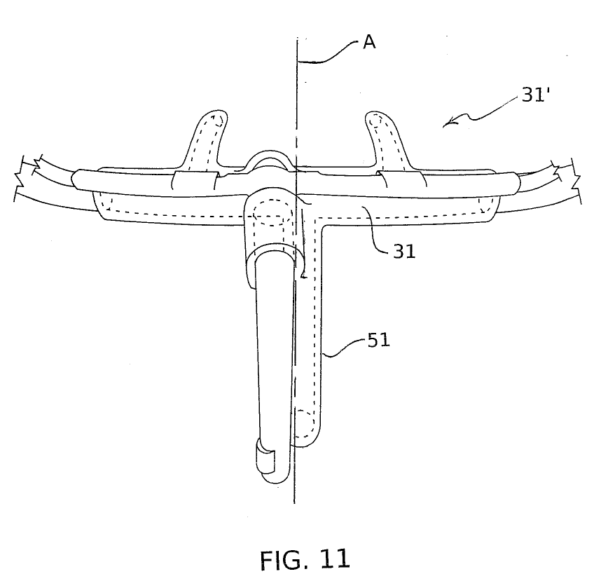

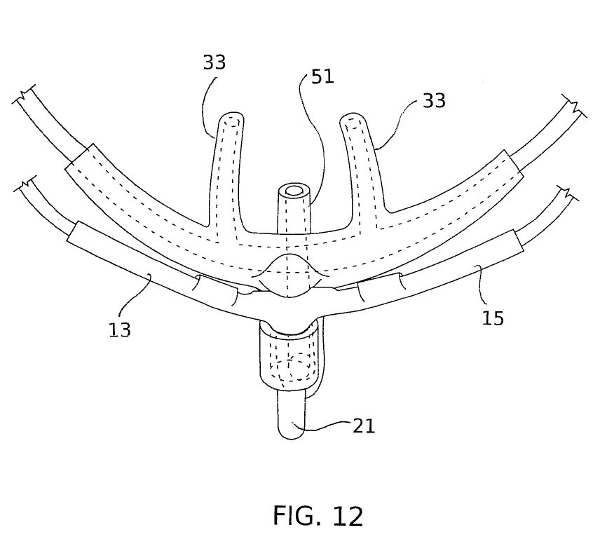

Observing FIGS. 9-12 is a further embodiment of the present invention which includes the holster 41 and stop portion 43 in combination with a cannula 31′ having an oral airflow pressure sensing tube 51 also communicating with the main body of the cannula 31′ in addition to the nasal prongs 33. The oral pressure sensing tube 51 is provided to be substantially centered on, or even slightly offset relative to the centerline A of the cannula body and the nasal prongs 33 on the cannula 31′. In order to ensure that the oral sensing thermistor 7 is not blocked in any manner by the oral pressure sensing tube 51, the holster 41 and stop 43 in this embodiment is radially offset from both the cannula centerline A as well as a centerline of the oral pressure sensing tube as in FIG. 11. This offset separation ensures that when the lower branch 21 of the sensor 1 is inserted through and into the holster 41, the lower branch 21 extends along the side of the oral pressure sensing tube 51 and thus can be directly aligned adjacent the patients oral airflow without being blocked by the pressure sensing tube 51.

Similar to the description of the first embodiment in FIGS. 5, 6 and 7, the holster 41 in FIGS. 9, 10 and 11 is provided with a sensor passage 47 and an upper stop 43 to define the notch 45 therebetween into which the center joint of the pressure sensor 1 is placed when the temperature sensor 1 is combined with the oral and nasal pressure sensing cannula 31′ to create the diagnostic system as shown and described herein.

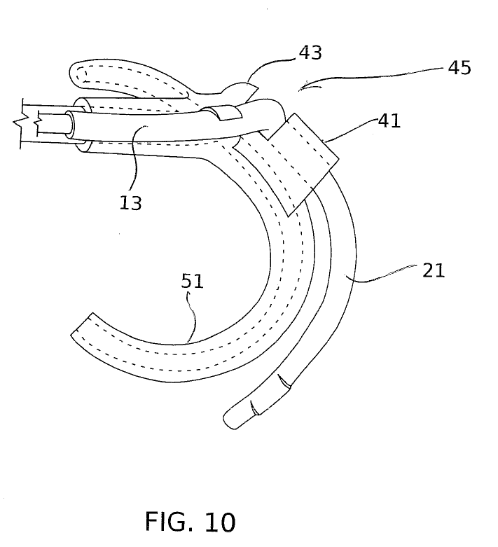

For the apparatus and system as described above, the temperature sensor 1 and the pressure sensing cannula 31, 31′ can be used together and facilitate obtaining similar but differently processed signals indicative of the patients breathing patterns. The malleability and adjustability of the T-shaped pressure sensor ensures that the left and right upper branches 13, 15 can be modified in any manner so that they essentially align with the nasal prongs 33 and the nares of the patients nostrils. The relative flexibility allows the left and right upper branches 13, 15 as well as the lower branch 21 to be bent inwards or outwards so as to conform to a bend in the cannula body, for instance, as seen in FIGS. 5 and 9 while as seen in FIG. 10 the lower branch 21 may be bent so as to achieve an entirely different axial and radial curvature and/or alignment than the oral sensing tube as seen in FIG. 10. For example, the free end of lower branch 21 may be moved in any 360° range relative to a free end of the pressure sensing tube and so can more accurately be placed in the direct airflow of the patients mouth. Relative to the pressure sensing tube and therefore potentially provide a more accurate data from the patients respiratory airflow.

Since certain changes may be made in the above described improved sleep apnea diagnosing apparatus and method, without departing from the spirit and scope of the invention herein involved, it is intended that all of the subject matter of the above description or shown in the accompanying drawings shall be interpreted merely as examples illustrating the inventive concept herein and shall not be construed as limiting the invention.

Wherefore, I/we claim:

1. A system for determining a breathing cycle of a patient, the system comprising:
a first sensing device for sensing pressure during the breathing cycle of the patient;
a second sensing device for sensing temperature of the patients inspiration and expiration during the breathing cycle concurrently with the pressure; and
wherein the system provides an output signal indicating the concurrently measured pressure and temperature of the patients breathing cycle.

2. The system as set forth in claim 1 wherein the first sensing device for sensing pressure comprises a cannula for receiving a sample of expiration flow from the patient and the second sensing device for sensing temperature further comprises at least a thermistor to be located in the patients inspiration and expiration flow path.

3. The system as set forth in claim 2 wherein the cannula further comprises an integral holster which directly secures an intermediate portion of the second sensing device relative to the cannula to facilitate locating the thermistor in a desired region of the patients inspiration and expiration flow path.

4. The system as set forth in claim 3 wherein the second sensing device comprises a plurality of branches extending from the intermediate portion of the second sensing device and wherein each branch extends from the intermediate portion of the second sensing device to a free end.

5. The system as set forth in claim 4 further comprising a thermistor being positioned in at least one of the plurality of extending branches of the second sensing device and the thermistor being connected in a circuit for determining temperature change due to the patients inspiration and expiration.

6. The system as set forth in claim 5 wherein the circuit in which the thermistor is connected includes a test circuit comprising a switch having a first state in which the switch is open and a second state in which the switch is closed and the test circuit indicates the continuity of the thermistor circuit.

7. The system as set forth in claim 5 wherein each branch of the temperature sensing device is independently manipulatable relative to any other branch to ensure that the thermistor can be effectively positioned in the respiratory airflow of the patient.

8. The system as set forth in claim 5 wherein the plurality of branches comprise a first nasal branch and a second nasal branch extending in substantially opposite directions from the intermediate portion of the second sensing device to a respective first free end and a second free end and an oral branch extending substantially perpendicular to the first and second nasal branches from the intermediate portion of the second sensing device to a third free end.

9. The system as set forth in claim 8 wherein at least one of the first nasal branch and the second nasal branch includes a nasal thermistor and the oral branch includes an oral thermistor for measuring a change in temperature of both the nasal and oral inspiration and expiration of the patient.

10. The system as set forth in claim 3 wherein the integral holster on the cannula comprises a passage for receiving and supporting at least one of the plurality of branches of the second sensing device.

11. The system as set forth in claim 10 wherein the passage for receiving and supporting at least one of the plurality of branches of the second sensing device is substantially equidistant space between the nasal prongs.

12. The system as set forth in claim 10 wherein the cannula comprises a first and second nasal prongs for communicating with the nares of the patient and an oral prong having an oral flow passage for communicating with the oral inspiration and expiration of the patient is positioned along a central plane spaced equidistant between the first and second nasal prongs.

13. The system as set forth in claim 12 wherein the passageway defined by the holster which receives and supports a branch of the second sensing device is substantially parallel with at least a portion of the oral flow passage of the oral prong.

14. The system as set forth in claim 13 wherein the passageway defined by the holster which receives and supports a branch of the second sensing device is offset from the central plane spaced equidistant between the first and second nasal prongs.

15. A method for concurrently measuring both airway pressure and airflow temperature of a patient and determining a breathing cycle of the patient, the method comprising the steps of:
sensing pressure during the breathing cycle of the patient;
sensing temperature of the patients inspiration and expiration during the breathing cycle concurrently with the pressure; and
derive a signal from the sensed pressure and temperature indicative of the concurrently measured pressure and temperature of the patients breathing cycle.

16. The method for concurrently measuring both airway pressure and airflow temperature of a patient and determining a breathing cycle of the patient as set forth in claim 15 further comprising the step of providing a cannula for receiving a sample of expiration flow from the patient and a temperature sensor located in the patients inspiration and expiration flow path for sensing temperature.

17. The method for concurrently measuring both airway pressure and airflow temperature of a patient and determining a breathing cycle of the patient as set forth in claim 16 further comprising the step of defining an integral holster on the cannula which directly receives and secures an intermediate portion of the temperature sensor relative to the cannula to facilitate locating the temperature sensor in a desired region of the patients inspiration and expiration flow path.

18. The method for concurrently measuring both airway pressure and airflow temperature of a patient and determining a breathing cycle of the patient as set forth in claim 17 further comprising the step of forming the temperature sensor having a plurality of branches extending from the intermediate portion of the temperature sensor to a free end spaced from the intermediate portion.

19. The system as set forth in claim 18 further comprising a thermistor being positioned in each of the plurality of extending branches of the second sensing device and the thermistors being connected in a circuit for determining temperature change due to the patients inspiration and expiration.

20. The method for concurrently measuring both airway pressure and airflow temperature of a patient and determining a breathing cycle of the patient as set forth in claim 15 further comprising the step of attaching a plurality of temperature sensors connected in a common circuit to a nasal and oral cannula such that the temperature sensors can be independently positioned relative to the nasal and oral cannula directly at the outlet of the nares of the patients nose and adjusted to properly position each temperature sensor in a manner to prevent contact of the sensor with the patients skin.

21. A temperature sensing device for determining the temperature change in a patients oral and nasal inspiration and expiration during a breathing cycle, the temperature sensing device comprising at least a first nasal thermistor and an oral thermistor for determining a temperature change in a patients breathing; and
wherein each thermistor is supported by a respective branch of the temperature sensing device connected at a common end point and extending radially outwardly from the common end point to a distal end.

22. The temperature sensing device for determining the temperature change in a patients oral and nasal inspiration and expiration during a breathing cycle as set forth in claim 21 wherein the common end point is held in a substantially fixed orientation relative to the patients face, nares and mouth and the branches of the temperature sensing device are independently positionable relative to the common end point and the patients face, nares and mouth.

23. The temperature sensing device for determining the temperature change in a patients oral and nasal inspiration and expiration during a breathing cycle as set forth in claim 21 wherein a circuit is connected to the nasal and oral thermistor for conditioning a signal generated by the oral and nasal thermistors and the circuit comprises a test circuit for indicating the continuity of the circuit when a switch is closed.

24. The temperature sensing device for determining the temperature change in a patients oral and nasal inspiration and expiration during a breathing cycle as set forth in claim 23 wherein the circuit further comprises a first lead and a second lead extending from at least one of the free ends of the branches of the temperature sensing device.

25. The temperature sensing device for determining the temperature change in a patients oral and nasal inspiration and expiration during a breathing cycle as set forth in claim 21 further comprising a first nasal branch extending from the common end point to a free end and an oral branch extending from the common end point to a second free end and the first nasal branch and oral branch being connected at the common end point in a substantially perpendicular manner.

26. The temperature sensing device for determining the temperature change in a patients oral and nasal inspiration and expiration during a breathing cycle as set forth in claim 25 wherein the first nasal branch and the oral branch are flexibly manipulatable about the common end point into a relative angle other than perpendicular aligned.

27. The temperature sensing device for determining the temperature change in a patients oral and nasal inspiration and expiration during a breathing cycle as set forth in claim 25 wherein the first nasal branch and the oral branch are flexibly manipulatable about the common end point independently of one another.
